(19) United States
(12) Patent Application Publication
(10) Pub. No.: US 2026/0263487 A1
(43) Pub. Date: Sep. 10, 2026

(54) USE OF CANNABIDIOL IN THE TREATMENT OF EPILEPSY

(71) Applicant: Jazz Pharmaceuticals Research UK Limited, Sittingbourne (GB)

(72) Inventors: Jie LI, Carlsbad, CA (US); Kevin James Craig, Sittingbourne (GB); Daniel Adam Checketts, Sittingbourne (GB); David John Critchley, Sittingbourne (GB)

(21) Appl. No.: 19/669,655

(22) Filed: May 6, 2026

Related U.S. Application Data
(63) Continuation of application No. 18/002,437, filed on Jun. 16, 2021, now Pat. No. 12,661,365.

(30) Foreign Application Priority Data
Jun. 18, 2020 (GB) 2009321.7

(51) Int. Cl. A61K 31/00; A61K 31/522

(52) U.S. Cl. CPC A61K 31/658; A61K 31/522

(57) ABSTRACT

The present invention relates to the use of cannabidiol (CBD) in the treatment of patients with childhood-onset epilepsy who are concurrently taking caffeine. Where the CBD is used in combination with caffeine, caution should be taken. For example, the dose of either the CBD and/or caffeine may be required to be reduced. Moreover, the patient may need to be monitored for side effects of said drug-drug interaction. Preferably the CBD used is in the form of a highly purified extract of cannabis such that the CBD is present at greater than 95% of the total extract (w/w) and the other components of the extract are characterised. In particular the cannabinoid tetrahydrocannabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

### FIELD OF THE INVENTION

The present invention relates to the use of cannabidiol (CBD) in the treatment of patients with childhood-onset epilepsy who are concurrently taking caffeine.

Where the CBD is used in combination with caffeine, caution should be taken. For example, the dose of either the CBD and/or caffeine may be required to be reduced. Moreover, the patient may need to be monitored for side effects of said drug-drug interaction.

Preferably the CBD used is in the form of a highly purified extract of cannabis such that the CBD is present at greater than 95% of the total extract (w/w) and the other components of the extract are characterised. In particular the cannabinoid tetrahydrocannabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

### BACKGROUND TO THE INVENTION

The cannabis plant (Cannabis sativa L.) produces trichomes that synthesize a large number of pharmacologically active compounds called phytocannabinoids. The most abundant of these are THC and CBD, although the amounts and proportions of the various phytocannabinoids in each plant vary by strain and can be adjusted by breeding.

Epidiolex is a liquid formulation of botanically derived, highly purified CBD extract that has been developed for use as a treatment for various orphan paediatric epilepsy syndromes, characterised in that the patients are deemed to be treatment-resistant on one or more anti-epileptic drugs (AEDs) (see WO 2019/97238 and WO 2016/203239). The drug has been approved for the treatment of seizures associated with Dravet syndrome and Lennox-Gastaut syndrome. It is formulated from extracts prepared from Cannabis sativa L. plants that have a defined chemical profile and contain consistent levels of CBD as the principal phytocannabinoid. Extracts from these plants are processed to yield pure (>95 to >98% w/w) CBD. The pure CBD is subsequently dissolved in excipients with added sweetener and flavouring.

Due to the above-outlined use of CBD in medicine, this has necessitated understanding different interactions CBD may have with other medicines or drugs. Drug-drug interactions (DDIs) are one of the commonest causes of medication error and can both induce the development of adverse drug reactions or reduce the clinical efficacy.

Caffeine is the world's most widely consumed psychoactive drug. It is a central nervous system stimulant of the methylxanthine class. It is found in the seeds, nuts and leaves of a number of different plants, including: Coffea Arabica (used for coffee), Thea sinensis (used for tea), Cola acuminata (used as a nut, tea or in soft drinks including cola), Theobroma cacao (used in cocoa and chocolate) and Paulinia cupana (used as guarana in snack bars and energy drinks) 1. Effects of caffeine include feeling more alert and active, as well as being more restless and excitable. It was reported in a 2010 Pediatrics journal study that a significantly high percentage of children, 75%, consume caffeine on a daily basis². Such a high percentage means that it is vital to understand the effects of caffeine and CBD co-consumption, as there may be a significant overlap between children consuming caffeine and young patients being administered their prescribed CBD-based medication.

Previous anecdotal reports have suggested synergistic effects of CBD and caffeine. The authors of these reports claim that the combination of CBD and caffeine can remove the jittery edge that a caffeine hit often brings, as well as feelings of anxiety and nausea^{3,4}. In fact, several companies have commercialized CBD-infused coffee, for example Green Roads CBD Coffee and Tea⁵ and Subduction Coffee+Hemp⁶. However, such commercially available supplies and anecdotal reports use low doses of CBD (~30 mg per serving), not nearly as high as the therapeutic doses in Epidiolex used to treat Dravet Syndrome and Lennox-Gastaut Syndrome. Epidiolex is prescribed for use at doses of 10-20 mg/kg/day, therefore a 40 kg child may be taking doses of up to 800 mg/day. Clearly in a heavier child or adolescent even higher doses such as 1500 mg/day could be taken.

The most common types of DDIs involve the inhibition or induction of one or more drug-metabolizing enzymes by a drug. When inhibitors or inducers of a particular drug-metabolizing enzyme are coadministered with a drug that is metabolized by that enzyme, the pharmacokinetic parameters of one or both drugs change, leading to increased or decreased drug exposures. It is this change in exposure that may result in adverse events, depending on the level of exposure, and doses may have to be changed. Dosing modifications are unpredictable because reducing a dose too much risks undertreating the patients, but over-dosing can increase exposure to potentially dangerous levels.

Elevated levels of CBD are known to cause transaminase elevation, rash, somnolence, sedation, lethargy, diarrhoea, pyrexia, weight decrease, nasopharyngitis, irritability, oropharyngeal pain, and decreased appetite. Transaminase elevations can lead to hepatic dysfunction, including unexplained nausea, vomiting, right upper quadrant abdominal pain, fatigue, anorexia, or jaundice and/or dark urine. Elevated exposure of caffeine exposure is known to cause insomnia, nervousness and restlessness, stomach irritation, nausea and vomiting, increased heart rate and respiration, and other side effects. The present disclosure reduces the incidence of one or more of the above side effects.

The pharmacological properties of CBD are not fully elucidated. As indicated on the FDA approved label for EPIDIOLEX®, CBD is metabolized in the liver and the gut by CYP2C19 and CYP3A4 enzymes, and UGT1A7, UGT1A9, and UGT2B7 isoforms. CBD inhibits uridine 5′-diphospho-glucuronosyltransferase (UGT) enzymes UGT1A9 and UGT2B7. CBD is reported to be an inhibitor of CYP2B6, CYP2C8, CYP2C9, and CYP2C19. Data also suggest that CBD inhibits CYP3A4. CBD may induce or inhibit CYP1A2 and CYP2B6 at clinically relevant concentrations. However, it is not known if CBD is a strong, moderate, or weak inhibitor, or indeed if CBD-mediated inhibition of any of these enzymes produces a clinically significant result. Samanta (2019) 7 further suggests CBD may induce or inhibit CYP1A2 activity, identifying caffeine as a substrate of CYP1A2, but does not present any evidence towards inhibition or induction. The present invention discloses clinically significant biochemical findings in several subjects during the trial: elevations in liver enzymes (ALT, AST, and GGT).

The present invention describes data from healthy subjects who received a single dose of caffeine after having taken repeated doses of CBD during an open-label, Phase 1 clinical trial. It was found that CBD increased the levels of caffeine in the subject's blood. Such an interaction is unexpected and as such the use of these drugs in combination should be done with close monitoring of the patient.

Furthermore, in the same study it was observed that administration of CBD in doses commonly administered to DS and LGS patients (approx. 20 mg/kg/day) to healthy adults resulted in elevations in liver chemistries consistent with drug induced liver injury (DILI). Such a finding was surprising as such high elevations in healthy volunteers has previously not been observed.

### BRIEF SUMMARY OF THE DISCLOSURE

In accordance with a first aspect of the present invention there is provided cannabidiol (CBD) for use in the treatment of childhood-onset epilepsy in patients who are concurrently taking caffeine characterised in that the blood levels of caffeine and associated markers are monitored to ensure the levels do not become toxic.

In a further embodiment of the invention the blood levels of liver enzymes are additionally monitored. These enzymes ALT, AST, and GGT are markers associated with drug induced liver injury (DILI). Higher than normal levels of liver enzymes may be present when CBD and caffeine are coadministered.

Preferably, the dose of CBD is lowered. Alternatively, the dose of caffeine is lowered. More preferably the dose of CBD and caffeine are lowered.

Preferably, the CBD is in the form of a highly purified extract of cannabis which comprises at least 95% (w/w) CBD, which comprises less than 0.15% THC and up to 1% CBDV. Alternatively, the CBD is present as a synthetic compound.

Preferably, the lowered dose of CBD ranges from about 5 mg/kg/day to about 20 mg/kg/day. Alternatively, the dose of caffeine is lowered to below 200 mg/day.

An average cup of coffee comprises approximately 95 mg of caffeine and therefore a patient may inadvertently consume doses of caffeine which exceed safe levels without realizing and as such blood levels should be monitored during treatment.

Preferably, the childhood-onset epilepsy is: Lennox-Gastaut Syndrome; Myoclonic Absence Epilepsy; Tuberous Sclerosis Complex; Dravet Syndrome; Doose Syndrome; Jeavons Syndrome; CDKL5; Dup15q; Neuronal ceroid lipofuscinoses (NCL) and brain abnormalities.

In accordance with a second aspect of the present invention there is provided a method of treating childhood-onset epilepsy in an individual in need thereof, comprising administering to the patient a therapeutically effective amount of cannabidiol with caution, wherein the individual is taking caffeine concurrently.

Preferably the said caution comprises lowering the dose of cannabidiol. Alternatively, the said caution comprises lowering the dose of caffeine.

Preferably the said caution comprises monitoring said individual for side effects.

More preferably the said caution further comprises discontinuing cannabidiol if said side effects are observed.

More preferably still the said caution comprises advising said individual of side effects from said concurrent therapy.

### BRIEF DESCRIPTION OF THE DRAWINGS

Embodiments of the invention are further described hereinafter with reference to the accompanying drawings, in which:

FIG. 1 shows Geometric Mean Plasma Concentrations of Caffeine Following Administration of Caffeine+Placebo (Day 1) and of Caffeine+CBD (Day 26) on a linear scale.

FIG. 2 shows Geometric Mean Plasma Concentrations of Paraxanthine Following Administration of Caffeine+Placebo (Day 1) and of Caffeine+CBD (Day 26) on a linear scale.

FIG. 3 shows Geometric Mean and Individual Subject Cmax for Caffeine and Paraxanthine Following Administration of Caffeine+Placebo (Day 1) and of Caffeine+CBD (Day 26).

FIG. 4 shows Geometric Mean and Individual Subject AUC0-∞ for Caffeine and Paraxanthine Following Administration of Caffeine+Placebo (Day 1) and of Caffeine+CBD (Day 26).

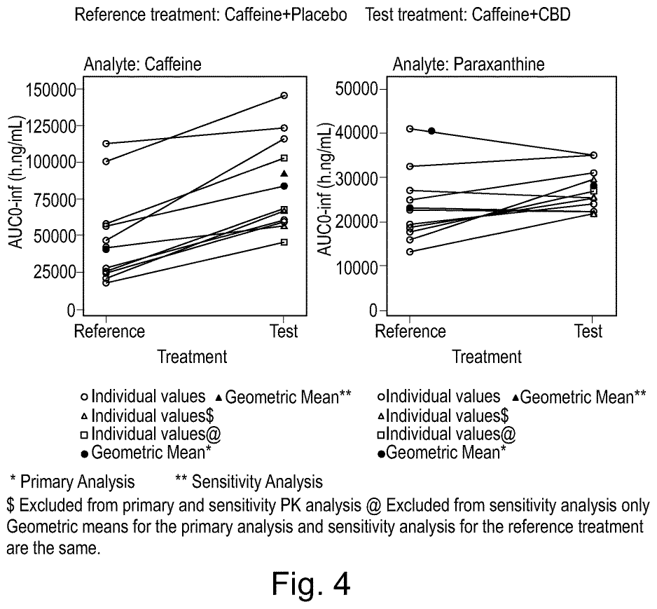

FIG. 5 shows Geometric LS Mean Ratio and 90% CI Showing the Effect of Steady-state CBD on Exposure to Caffeine and Paraxanthine.

FIG. 6 shows a Graph of serial liver chemistries for the 5 participants with ALT ≥5×ULN.

### DEFINITIONS

Definitions of some of the terms used to describe the invention are detailed below:

The cannabinoids described in the present application are listed below along with their standard abbreviations.

TABLE 1
Cannabinoids and their abbreviations

| | | |
|---|---|---|
| CBD | Cannabidiol | 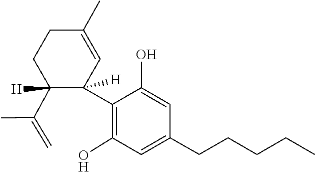 [SMILES: C=C(C)[C@@H]1CCC(C)=C[C@H]1c1c(O)cc(CCCCC)cc1O] |
| CBDA | Cannabidiolic acid | 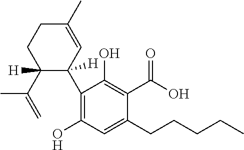 [SMILES: C=C(C)[C@@H]1CCC(C)=C[C@H]1c1c(O)cc(CCCCC)c(C(=O)O)c1O] |
| CBDV | Cannabidivarin | 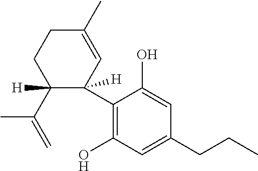 [SMILES: C=C(C)[C@@H]1CCC(C)=C[C@H]1c1c(O)cc(CCC)cc1O] |
| CBDVA | Cannabidivarinic acid | 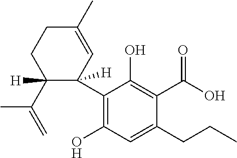 [SMILES: C=C(C)[C@@H]1CCC(C)=C[C@H]1c1c(O)cc(CCC)c(C(=O)O)c1O] |
| THC | Tetrahydrocannabinol | 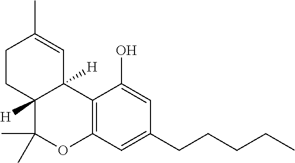 [SMILES: CCCCCc1cc(O)c2c(c1)OC(C)(C)[C@@H]1CCC(C)=C[C@@H]21] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoid extracts” are defined as cannabinoids that have been extracted from the cannabis plant and purified to the extent that other cannabinoids and non-cannabinoid components that are co-extracted with the cannabinoids have been substantially removed, such that the highly purified cannabinoid is greater than or equal to 95% (w/w) pure. The highly purified cannabinoid extract may be purified further such that the cannabinoid content is greater than or equal to 98% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Childhood epilepsy” refers to the many different syndromes and genetic mutations that can occur to cause epilepsy in childhood. Examples of some of these are as follows: Dravet Syndrome; Myoclonic-Absence Epilepsy; Lennox-Gastaut syndrome; Generalized Epilepsy of unknown origin; CDKL5 mutation; Aicardi syndrome; tuberous sclerosis complex; bilateral polymicrogyria; Dup15q; SNAP25; and febrile infection related epilepsy syndrome (FIRES); benign rolandic epilepsy; juvenile myoclonic epilepsy; infantile spasm (West syndrome); and Landau-Kleffner syndrome. The list above is non-exhaustive as many different childhood epilepsies exist.

Cₘₐₓ is the maximum observed plasma concentration.

tₘₐₓ is the time to attain maximum observed plasma concentration.

AUC_{0-∞} is the area under the plasma concentration-time curve from time 0 to infinity, calculated as AUC_{0-∞}=AUC₀₋ₜ+Ċₗₐₛₜ/kₑₗ, where Ċₗₐₛₜ is the estimated last plasma concentration and where kₑₗ is the terminal phase rate constant.

AUC₀₋ₜ is the area under the plasma concentration-time curve up to time t, where t is the last point with a concentration above the lower limit of quantification (LLOQ).

Clinical laboratory measurements of liver injury and function, the Upper Limit of Normal (ULN) values used are: 68 international units [IU]/L for ALT, 45 IU/L for aspartate aminotransferase (AST), 129 IU/L for alkaline phosphatase (ALP), 29 μmol/L for total bilirubin (TBL), and 59 IU/L for gamma-glutamyl transferase (GGT).

Drug induced liver injury (DILI) is when serum ALT exceeds 5x ULN.

### DETAILED DESCRIPTION OF THE INVENTION

### Preparation of Highly Purified CBD Extract

The following describes the production of the highly-purified (>95-98% w/w) cannabidiol extract which has a known and constant composition was used in the Examples below.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 98% CBD. Although the CBD is highly purified because it is produced from a cannabis plant rather than synthetically there is a small number of other cannabinoids which are co-produced and co-extracted with the CBD. Details of these cannabinoids and the quantities in which they are present in the medication are as described in Table 2 below.

TABLE 2
Composition of highly purified CBD extract

| Cannabinoid | Concentration |
|---|---|
| CBD | >95-98% w/w |
| CBDA | NMT 0.15% w/w |
| CBDV | NMT 1.0% w/w |
| Δ⁹ THC | NMT 0.15% w/w |
| CBD-C4 | NMT 0.5% w/w |

>greater than
NMT—not more than

### Example 1: A Phase 1, Open-Label, Pharmacokinetic Drug-Drug Interaction Trial to Investigate the Effect of Cannabidiol on the Pharmacokinetics (PK) of Caffeine

Primary Objectives: To investigate the effect of CBD treatment following repeated dosing on the PK of a single dose of caffeine in healthy subjects.

Primary Endpoints: The primary PK parameters were: AUC_{0-∞}, AUC₀₋ₜ, Cₘₐₓ, and tₘₐₓ for caffeine. The PK parameter endpoints, derived from the plasma concentration-time profiles of caffeine on Day 1 administered with placebo and the PK parameter endpoints derived from a single dose of caffeine in participants at steady state CBD following 13 days of CBD, 20 mg/kg twice daily (b.i.d).

Secondary Objectives: To evaluate the safety and tolerability of CBD when given with a single dose of caffeine in healthy subjects.

Secondary Endpoints: Safety includes: incidence and severity of adverse events (AEs), incidence of laboratory abnormalities based on hematology, clinical chemistry, and urinalysis test results; 12-lead electrocardiogram (ECG) parameters, vital sign measurements, physical examinations, Columbia-Suicide Severity Rating Scale (C-SSRS) questionnaire scores; the PK parameter endpoints, derived from the plasma concentration-time profiles of caffeine on Day 1 administered with placebo and the PK parameter endpoints derived from a single dose of caffeine in participants at steady state CBD following 13 days of CBD, 20 mg/kg twice daily (b.i.d).

Design: This was a phase 1, open-label, single site trial to investigate the effect of multiple dose administration of CBD on the PK of caffeine in healthy subjects. The duration of the trial was approximately 10 weeks, which includes a screening period (up to 4 weeks), a treatment period (4 weeks) and a safety follow-up period (2 weeks). After signing the informed consent form (ICF), participants entered the screening period (Day-28 to-1). On Day-1, which is the day prior to Day 1, the first day of IMP (caffeine+placebo) administration, screened participants who continued to meet eligibility criteria were admitted to the clinical research unit (CRU). The subjects were resident in the CRU for 2 periods. Subjects were administered a concurrent dose of 7.5 mL of placebo oral solution and 200 mg caffeine on Day 1, 30 minutes after starting a standardized breakfast. They were discharged on Day 3 after completion of the assessments. On Day 3, the first dose of CBD was taken in the morning in the CRU, and on this day, the escalating doses of CBD were dispensed to subjects to be taken at home from Day 4 to Day 12. After discharge on Day 3, the subjects returned to the CRU for ambulatory visits on Days 12, 18, and 23. At these ambulatory visits, the maintenance doses of CBD were dispensed to subjects to be taken at home from Day 13 to Day 25 (the evening dose of Day 25 was taken in the CRU). The subjects were admitted again to the CRU in the afternoon of Day 25. On Days 26 and 27, subjects received CBD b.i.d, and on Day 26, a single oral dose of caffeine was given concurrently with the morning dose of CBD. The subjects were discharged on Day 28 after completion of the assessments. The subjects had a follow-up visit 14 to 16 days after the last IMP dose. In addition to the scheduled follow-up visit, several subjects came back to the CRU after Day 28 for unscheduled visits for additional blood sampling regarding out-of-normal reference range values of liver enzymes. CBD and caffeine were administered as shown in Table 3.

TABLE 3
Treatment Schedule

| Day | Morning Dose | Evening Dose |
|---|---|---|
| 1 | 7.5 mL placebo oral | Not applicable |
| | solution (matched to CBD) | |
| | 200 mg caffeine oral tablet | |
| 3 | 250 mg CBD: 2.5 mL oral | Not applicable |
| | solution | |
| 4-5 | 250 mg CBD: 2.5 mL oral | 250 mg CBD: 2.5 mL oral |
| | solution | solution |
| 6-7 | 500 mg CBD: 5 mL oral | 250 mg CBD: 2.5 mL oral |
| | solution | solution |
| 8-9 | 500 mg CBD: 5 mL oral | 500 mg CBD: 5 mL oral |
| | solution | solution |
| 10-11 | 750 mg CBD: 7.5 mL oral | 500 mg CBD: 5 mL oral |
| | solution | solution |
| 12-25 | 750 mg CBD: 7.5 mL oral | 750 mg CBD: 7.5 mL oral |
| | solution | solution |
| 26 | 750 mg CBD: 7.5 mL oral | 750 mg CBD: 7.5 mL oral |
| | solution | solution |
| | 200 mg caffeine oral tablet | |
| 27 | 750 mg CBD: 7.5 mL oral | 750 mg CBD: 7.5 mL oral |
| | solution | solution |

Formulation Mode of Administration, Dose, Regimen: The CBD formulation is an oral liquid formulation that is clear and colourless to yellow in appearance (100 mg/mL CBD in sesame oil with anhydrous ethanol, added sweetener (sucralose), and strawberry flavouring. The oral liquid formulation is administered with a syringe. The CBD formulation is taken b.i.d. 30 minutes after starting a standard meal. In this trial, a maximum dose of 750 mg CBD b.i.d was selected, considered a therapeutic dose in epilepsy patients.

The placebo is an oral liquid formulation (sesame oil and anhydrous ethanol with added sweetener [sucralose], and strawberry flavouring). The oral liquid formulation was administered with a syringe.

The caffeine is provided as a 50 mg tablet.

On Day 1 and Day 26, the IMP (caffeine and placebo on Day 1, and caffeine and CBD on Day 26) were taken between 08:00 hours and 09:00 hours. From Day 3 to Day 27, the morning dose of CBD was taken approximately at 08:00 h and the evening dose (not applicable on Day 3) was taken 12 hours later. Dosing for each individual subject was to be at around the same time (±1 hour) on each dosing day. The time of the morning dose on Day 26 matched the time of the morning dose on Day 1 (with a margin of ±5 minutes).

The use of all prescribed medication and all over-the-counter medication, vitamin preparations and other food supplements, or herbal medications was prohibited from first admission to the CRU until the follow-up visit. The use of methylxanthine-containing beverages or food (coffee, [iced] tea, cola, chocolate [milk], mocha drinks/sweets, energy drinks) was not allowed from first admission to the CRU until discharge on Day 28. Foods and beverages containing grapefruit, Seville oranges, pomelos, star fruit, or cranberries, or cruciferous vegetables were not allowed from first admission to the CRU until discharge on Day 28. Alcohol was not allowed from 48 hours prior to each admission to the CRU and throughout the inpatient period, and from 48 hours prior to the safety follow-up visit. Strenuous exercise was not allowed from 7 days prior to first admission to the CRU until the follow-up visit. Subjects were not to consume any foods containing poppy seeds within 72 hours (3 days) prior to each admission to the CRU as this could cause a false positive drug screen result. The use of tobacco- or nicotine-containing products was not allowed from first admission to the CRU until the follow-up visit.

Pharmacokinetic Assessments: Plasma concentrations of caffeine and its metabolite paraxanthine were determined on the following days: 1, 2, 3, 26, 27 and 28. Plasma concentration of CBD determined on the following days: 23, 25, and 26. These were determined using liquid chromatography and tandem mass spectrometry.

The following assessments were performed: demographics, medical history, physical examination, C-SSRS, vital signs, body weight, height, 12-lead ECG, adverse events (AEs), previous and concomitant medications recorded. Clinical laboratory samples including chemistry, hematology, serology, urine drug screen, alcohol test.

Trial subjects were sixteen healthy male and female subjects aged between 18 and 60. All 16 participants took at least 1 dose of trial drug; 9 (56%) completed treatment as planned and received the expected total dose of 400 mg caffeine and 31.25 g cannabidiol.

### Results

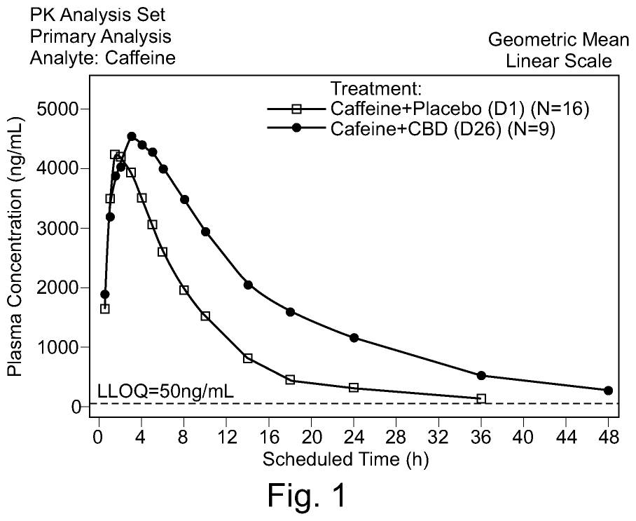

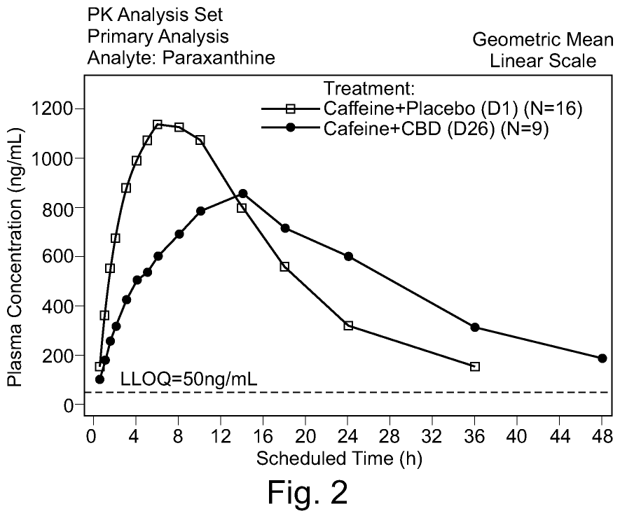

Plasma concentrations of caffeine and paraxanthine following administration of caffeine+placebo and of caffeine+CBD are presented in FIGS. 1 and 2.

Trough plasma samples for CBD were taken on Days 23, 25, and 26. Trough levels of CBD confirmed that CBD had reached steady state before caffeine and CBD were coadministered on Day 26.

After dosing with 200 mg caffeine and placebo on Day 1 and coadministration of 200 mg caffeine and 750 mg CBD on Day 26, caffeine and its metabolite paraxanthine were quantifiable in the majority of subjects at the first sampling time point, i.e., 0.5 hours.

On Day 1, following administration of 200 mg caffeine and placebo, maximum postdose geometric mean plasma concentrations were reached at 1.5 hours for caffeine and 6.0 hours for paraxanthine. On Day 26, following coadministration of 200 mg caffeine and 750 mg CBD, maximum postdose geometric mean plasma concentrations were reached at 3.0 hours for caffeine and 14.0 hours for paraxanthine.

As is apparent from the individual and geometric mean profiles, the elimination phase of caffeine and paraxanthine was multiphasic following 200 mg caffeine and 750 mg CBD coadministration on Day 26, and caffeine concentrations were higher and paraxanthine concentrations lower compared with administration of 200 mg caffeine and placebo on Day 1.

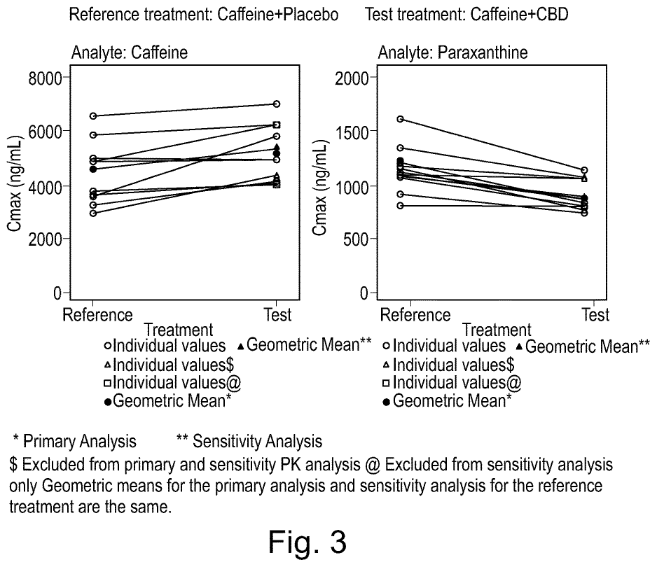

The PK parameters for caffeine and paraxanthine are presented in Table 4. The differences between geometric mean and individual subject PK parameters for caffeine and paraxanthine following administration of caffeine+placebo (Day 1) and of caffeine+CBD (Day 26) are shown in FIG. 3 (Cₘₐₓ) and 4 (AUC0-∞), and the statistical analyses of the differences in PK parameters are presented in Table 5.

TABLE 4
Summary of the Pharmacokinetic Parameters (Geometric Mean [Geometric CV %])
of Caffeine and Paraxanthine Following Administration of Caffeine + Placebo
(Day 1) and of Caffeine + CBD (Day 26)

| | Primary Analysis | | Sensitivity Analysis |
|---|---|---|---|
| | Caffeine + Placebo | Caffeine + CBD | Caffeine + CBD |
| | (Day 1) | (D 26-FU) | (D 26-FU) |
| Parameter | (N = 16) | (N = 9) | (N = 6) |

Caffeine

| | | | | | | |
|---|---|---|---|---|---|---|
| AUC₀₋ₜ (ng · h/mL) | 40000 | (56.7) | 78300 | (37.2) | 85500 | (35.1) |
| AUC_{0-∞} (ng · h/mL) | 40900 | (57.6) | 83400 | (41.0) | 92300 | (39.4) |
| Cₘₐₓ (ng/mL) | 4600 | (23.5) | 5170 | (21.1) | 5430 | (19.0) |
| tₘₐₓ^{a} (h) | 1.51 | (0.50-3.00) | 3.00 | (0.50-5.00) | 3.00 | (0.50-4.13) |

Paraxanthine

| | | | | | | |
|---|---|---|---|---|---|---|
| AUC₀₋ₜ (ng · h/mL) | 21500 | (27.8) | 23900 | (12.8) | 23900 | (14.0) |
| AUC_{0-∞} (ng · h/mL) | 23000 | (30.2) | 27900 | (18.4) | 28100 | (20.1) |
| Cₘₐₓ (ng/mL) | 1230 | (20.6) | 885 | (15.8) | 894 | (17.1) |
| tₘₐₓ^{a} (h) | 7.99 | (4.00-18.02) | 14.00 | (5.97-18.00) | 14.00 | (10.00-18.00) |

FU = follow-up; max = maximum; min = minimum; PK = pharmacokinetic.
Note:
For the treatment of caffeine + placebo, the primary analysis and sensitivity analysis are equal.
^{a}Median (min-max).

TABLE 5
Statistical Analysis of the Pharmacokinetic Parameters of Caffeine and Paraxanthine
Following Administration of Caffeine + Placebo (Day 1) and of Caffeine + CBD (Day 26)

| | | | | | | Ratio Test/Reference | |
|---|---|---|---|---|---|---|---|
| | | Geometric LS Means | | | | | 90% CI |
| Analyte | PK Parameter | n | Reference | n | Test | Estimate | [Lower, Upper] |

Primary Analysis

| | | | | | | | |
|---|---|---|---|---|---|---|---|
| Caffeine | Cₘₐₓ^{a} (ng/mL) | 16 | 4600 | 9 | 5269 | 1.15 | [1.04, 1.26] |
| | AUC₀₋ₜ^{a} (ng · h/mL) | 16 | 39969 | 9 | 75237 | 1.88 | [1.56, 2.27] |
| | AUC_{0-∞}^{a} (ng · h/mL) | 16 | 40856 | 9 | 79718 | 1.95 | [1.62, 2.35] |
| | tₘₐₓ^{b} (h) | 9 | 1.52 | 9 | 3.00 | 0.58 | [0.01, 1.50] |
| Paraxanthine | Cₘₐₓ^{a} (ng/mL) | 16 | 1227 | 7 | 961 | 0.78 | [0.72, 0.86] |
| | AUC₀₋ₜ^{a} (ng · h/mL) | 16 | 21454 | 7 | 23579 | 1.10 | [0.96, 1.26] |
| | AUC_{0-∞}^{a} (ng · h/mL) | 16 | 22972 | 7 | 27038 | 1.18 | [1.03, 1.35] |
| | tₘₐₓ^{b} (h) | 7 | 8.00 | 7 | 14.00 | 3.49 | [0.48, 6.00] |

Sensitivity Analysis

| | | | | | | | |
|---|---|---|---|---|---|---|---|
| Caffeine | Cₘₐₓ^{a} (ng/mL) | 16 | 4600 | 6 | 5460 | 1.19 | [1.03, 1.36] |
| | AUC₀₋ₜ^{a} (ng · h/mL) | 16 | 39969 | 6 | 71265 | 1.78 | [1.39, 2.29] |
| | AUC_{0-∞}^{a} (ng · h/mL) | 16 | 40856 | 6 | 76272 | 1.87 | [1.45, 2.41] |
| | tₘₐₓ^{b} (h) | 6 | 2.25 | 6 | 3.00 | 0.57 | [0.00, 1.25] |
| Paraxanthine | Cₘₐₓ^{a} (ng/mL) | 16 | 1227 | 6 | 984 | 0.80 | [0.73, 0.88] |
| | AUC₀₋ₜ^{a} (ng · h/mL) | 16 | 21454 | 6 | 22526 | 1.05 | [0.93, 1.18] |
| | AUC_{0-∞}^{a} (ng · h/mL) | 16 | 22972 | 6 | 26023 | 1.13 | [0.99, 1.30] |
| | tₘₐₓ^{b} (h) | 6 | 8.00 | 6 | 14.00 | 3.99 | [−0.02, 7.00] |

CI = confidence interval; LS = least squares; PK = pharmacokinetic.
Note:
Reference, caffeine + placebo treatment; Test, caffeine + CBD treatment.
^{a}AUC and Cₘₐₓ, the interaction effect was explored using a mixed effect (analysis of variance) model with treatment as fixed factor and subject as a random effect.
^{b}tₘₐₓ, nonparametric Wilcoxon signed-rank test presenting the Hodges-Lehman estimate and 90% CI based on the Tukey method. Median, median of the difference, and approximate 90% CI for the difference are presented.

For caffeine, as shown by the point estimates for the treatment ratios (primary analysis) in Table 5, when compared with caffeine and placebo (Day 1), coadministration of caffeine and CBD (Day 26) resulted in a slight increase in Cₘₐₓ (1.15, 90% CI: [1.04, 1.26]) and a larger increase in AUC₀₋ₜ (1.88, 90% CI: [1.56, 2.27]) and AUC_{0-∞} (1.95, 90% CI: [1.62, 2.35]). The tₘₐₓ for caffeine was later after administration of caffeine and CBD (Day 26) compared with caffeine and placebo administration (Day 1) (difference Hodges-Lehman estimate: 0.58, 90% CI: [0.01, 1.50]).

For paraxanthine, as shown by the point estimates for the treatment ratios (primary analysis), when compared with caffeine and placebo (Day 1), coadministration of caffeine and CBD (Day 26) resulted in a decrease in Cₘₐₓ (0.78, 90% CI: [0.72, 0.86]) and a slight increase in AUC₀₋ₜ (1.10, 90% CI: [0.96, 1.26]) and AUC_{0-∞} (1.18, 90% CI: [1.03, 1.35]). The tₘₐₓ for paraxanthine tended to be later after administration of caffeine and CBD (Day 26) compared with caffeine and placebo administration (Day 1) (difference Hodges-Lehman estimate: 3.49, 90% CI: [0.48, 6.00]).

Similar results were seen for the primary and sensitivity analyses.

Thus, exposure to caffeine increased by 15% for Cₘₐₓ and 95% for AUC_{0-∞} when caffeine was given with steady-state CBD compared to when caffeine was given with placebo.

Exposure to the CYP1A2-mediated caffeine metabolite paraxanthine was impacted by CBD coadministration as evidenced by a 22% decrease in Cₘₐₓ and 18% increase in AUC_{0-∞}. The tₘₐₓ for paraxanthine was delayed when caffeine was coadministered with CBD, which may reflect a slower formation of the metabolite.

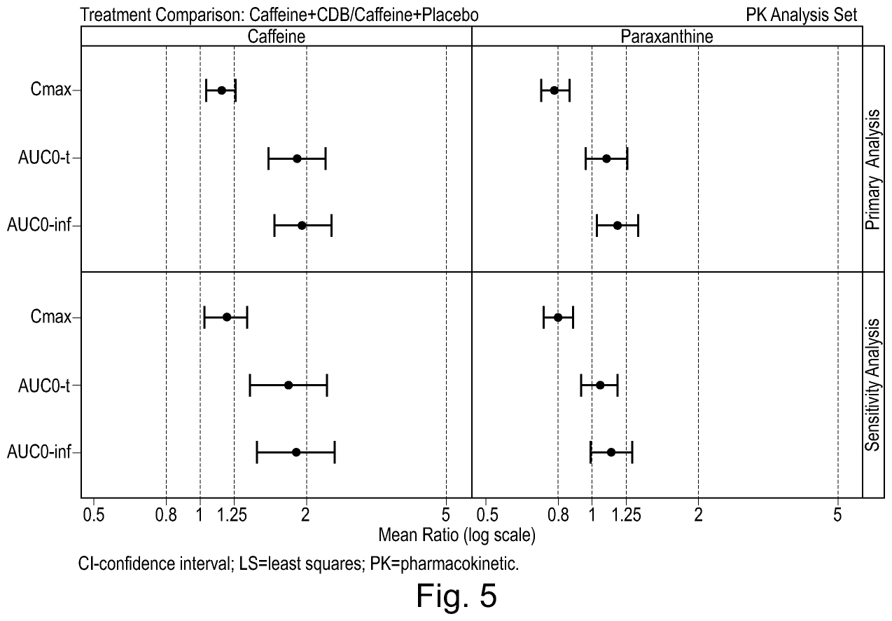

FIG. 5 provides a visual summary of the results of the primary PK endpoint for the trial, illustrating the point estimates for the ratio of the geometric LS means and the 90% CI for exposure to caffeine and paraxanthine when subjects were administered caffeine in the presence of steady-state CBD compared with administration of caffeine and placebo.

Coadministration of caffeine and CBD resulted in a change in caffeine and paraxanthine exposure (based on AUC₀₋ₜ, AUC_{0-∞}, and Cₘₐₓ). AUC₀₋ₜ was increased by 88% and 10% for caffeine and paraxanthine, respectively; AUC_{0-∞} was increased by 95% and 18% for caffeine and paraxanthine, respectively; and Cₘₐₓ was increased by 15% and decreased by 22% for caffeine and paraxanthine, respectively, when compared to administration of caffeine and placebo. Taken all together, CBD affects exposure of caffeine and its metabolite paraxanthine.

### Conclusions

Such findings delineate an important concern for epilepsy patients and also for the wider community with the increasing legalization of marijuana. This drug-drug interaction may have implications in epilepsy patients which are not correctly monitored over the course of their treatment.

Patients that are taking caffeine-containing drugs or products should be carefully monitored over the course of their treatment with CBD to ensure toxicity from prolonged caffeine exposure does not occur.

### Example 2: Liver Safety

In the above-outlined Phase 1 trial, abnormal liver chemistries were found for the subjects. This was unexpected as subjects were healthy adults receiving therapeutic equivalent doses of CBD used to treat Dravet Syndrome and Lennox-Gastaut Syndrome (1500 mg/day).

Measurements were performed at screening, on trial Days-1, 12, 18, 23, 27, and at follow-up.

### Results

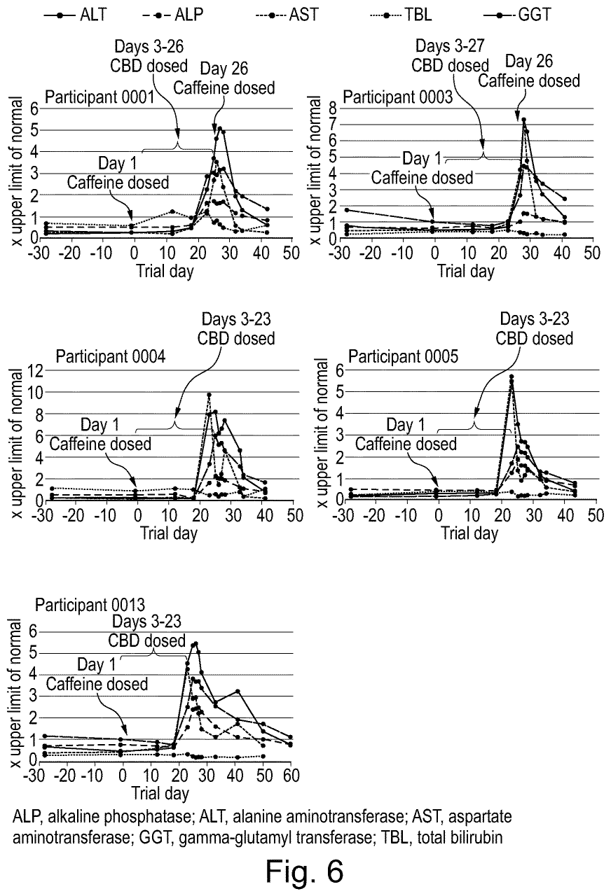

The most notable biochemical abnormalities were elevation in serum ALT, the related enzyme AST, and GGT. In 7 (44%) participants, peak serum ALT values were >ULN; in 6 (38%) participants the value was >2×ULN; and in 5 (31%) participants the peak ALT value was >5×ULN, the international consensus criteria for DILI (Table 6). The serial liver chemistries observed in the 5 participants with peak ALT values >5×ULN are displayed in FIG. 6.

All elevations occurred between 2-4 weeks exposure to cannabidiol, between days 18-27. Among the 6 participants with ALT elevations who were discontinued from the protocol, some had symptoms consistent with hepatitis, fever, or eosinophilia.

TABLE 6
Observed peak serum ALT and fold-changes from baseline value

| Reference | | | | | | |
|---|---|---|---|---|---|---|
| ULN | >ULN | ≥2 × ULN | ≥3 × ULN | ≥5 × ULN | ≥10 × ULN | ≥20 × ULN |
| Stated ULN | 7 (44%) | 6 (38%) | 5 (31%) | 5 (31%) | 0 (0) | 0 (0) |

### Conclusions

Therapeutic doses of cannabidiol administered to healthy adults can result in elevations in serum alanine aminotransferase consistent with drug-induced liver injury. Physicians should be alert to this potential effect from cannabidiol and be on the lookout for association with clinically important liver injury.

Furthermore, abnormal liver chemistries in healthy volunteers dosed with CBD at the doses used in this study have not heretofore been observed at the high rates seen in this trial (44%). As such the DDI observed between CBD and caffeine may have an impact on the DILI experienced by these patients.

It is therefore imperative that levels of CBD, caffeine, and liver enzymes (ALT, AST, and GGT) are measured in patients administered CBD who are concurrently taking caffeine either as a medication or recreationally. Caution is required to ensure DILI does not occur in these patients. Reduction of the dose of CBD, the dose of caffeine or the doses of both CBD and caffeine may be required to ensure patients liver chemistries are safe.

### REFERENCES

1. Brands, B., Sproule, B., & Marshman, J. (Eds.). (1998). Drugs & drug abuse (3rd ed.). Ontario: Addiction Research Foundation.
2. Warzak, W. J., Evans, S., Floress, M. T., Gross, A. C., Stoolman, S. (2010). Caffeine Consumption in Young Children. The Journal of Pediatrics. VOLUME 158, ISSUE 3, P508-509, Mar. 1, 2011. DOI:https://doi.org/10.1016/j.jpeds.2010.11.022
3. Gillen, D. (2019). How Does Caffeine Affect CBD? https://greendoorcbd.com/blogs/news/how-does-caffeine-affect-cbd#:~:text=it's%20widely%20reported%20by%20those,feelings%20of%20anxiety%20and%20nausea.
4. Malamut, M. (2019). I Drank CBD Coffee for a Week. Here's What It Did to My Anxiety. https://www.healthline.com/health t-cbd-coffee-anxiety#10
5. Green Roads CBD Coffee and Tea. https://greenroads.com/collections/cbd-tea-cbd-coffee?rfsn=2488702.aa938d
6. Subduction Coffee+Hemp. https://subductioncoffee.com/?afmc=2j&utm_campaign=2j&utm source=leaddyno&utm_medium=affiliate
7. Samanta (2019) “Cannabidiol: A Review of Clinical Efficacy and Safety in Epilepsy”, Pediatric Neurology, Vol. 96, 2019, pp. 24-29

1-18. (canceled)

19. A method of treating childhood-onset epilepsy in a patient in need thereof,
comprising administering to the patient a pharmaceutical composition comprising a cannabidiol (CBD) drug substance comprising at least 98% w/w CBD, wherein:
the patient is taking a CYP1A2 substrate concurrently; and
the dose of CYP1A2 substrate is reduced compared to a patient that is not taking CBD concurrently.

20. The method of claim 19, wherein the dose of CYP1A2 substrate is reduced to no more than 200 mg/day.

21. The method of claim 19, wherein the dose of CYP1A2 substrate is reduced to no more than 95 mg/day.

22. The method of claim 19, wherein the dose of CYP1A2 substrate is reduced to no more than 50 mg/day.

23. The method of claim 19, wherein the CYP1A2 substrate is caffeine.

24. The method of claim 19, wherein the dose of CBD is about 5 mg/kg/day to about 20 mg/kg/day.

25. The method of claim 19, wherein the dose of CBD is in the range of about 30 mg/day to about 1500 mg/day.

26. The method of claim 25, wherein the dose of CBD is in the range of about 250 mg/day to about 1500 mg/day.

27. The method of claim 25, wherein the dose of CBD is in the range of about 500 mg/day to about 1500 mg/day.

28. The method of claim 19, wherein the dose of CYP1A2 substrate is reduced to no more than 200 mg/day, and the dose of CBD is in the range of about 250 mg/day to about 1500 mg/day.

29. The method of claim 23, wherein the dose of CYP1A2 substrate is reduced to no more than 200 mg/day, and the dose of CBD is in the range of about 250 mg/day to about 1500 mg/day.

30. The method of claim 19, wherein the CBD drug substance comprises not more than 0.15% w/w CBDA.

31. The method of claim 19, wherein the CBD drug substance comprises at least 98% w/w CBD and not more than 1.0% w/w CBDV.

32. The method of claim 19, wherein the CBD drug substance comprises at least 98% w/w CBD and not more than 0.15% w/w A9THC.

33. The method of claim 19, wherein the CBD drug substance comprises at least 98% w/w CBD and not more than 0.5% w/w CBD-C4.

34. The method of claim 19, wherein the CBD drug substance comprises at least 98% w/w CBD, not more than 1.0% w/w CBDV, and not more than 0.15% w/w A9THC.

35. The method of claim 19, wherein the CBD drug substance comprises at least 98% w/w CBD, not more than 1.0% w/w CBDV, not more than 0.15% w/w A9THC, and not more than 0.5% w/w CBD-C4.

36. The method of claim 19, wherein the CBD drug substance comprises at least 98% w/w CBD, not more than 0.15% w/w CBDA, not more than 1.0% w/w CBDV, not more than 0.15% w/w A9THC, and not more than 0.5% w/w CBD-C4.

37. The method of claim 19, wherein the childhood-onset epilepsy is selected from the group consisting of Lennox-Gastaut Syndrome; Myoclonic Absence Epilepsy; Tuberous Sclerosis Complex; Dravet Syndrome; Doose Syndrome; Jeavons Syndrome; CDKL5; Dup15q; Neuronal ceroid lipofuscinoses (NCL) and brain abnormalities.

38. The method of claim 19, wherein the childhood-onset epilepsy is Lennox-Gastaut Syndrome.

39. The method of claim 19, wherein the childhood-onset epilepsy is Dravet Syndrome.

40. The method of claim 19, wherein the childhood-onset epilepsy is Tuberous Sclerosis Complex.

41. The method of claim 38, wherein the dose of CYP1A2 substrate is reduced to no more than 200 mg/day, and the dose of CBD is in the range of about 250 mg/day to about 1500 mg/day.

42. The method of claim 39, wherein the dose of CYP1A2 substrate is reduced to no more than 200 mg/day, and the dose of CBD is in the range of about 250 mg/day to about 1500 mg/day.

43. The method of claim 40, wherein the dose of CYP1A2 substrate is reduced to no more than 200 mg/day, and the dose of CBD is in the range of about 250 mg/day to about 1500 mg/day.
